(19) United States
(12) Patent Application Publication
(10) Pub. No.: US 2004/0241852 A1
(43) Pub. Date: Dec. 2, 2004

(54) Nucleic acid for a cloning vector

(76) Inventor: Reinhard Heiermann, Dortmund (DE)

Correspondence Address: HENRY M FEIEREISEN, LLC, 350 FIFTH AVENUE, SUITE 4714, NEW YORK, NY, 10118, US

(21) Appl. No.: 10/489,749

(22) PCT Filed: Sep. 16, 2002
(86) PCT No.: PCT/EP02/10375

(30) Foreign Application Priority Data
Sep. 16, 2001 (DE) 10145553.4

(51) Int. Cl. C12N 15/85; C07H 21/04

(52) U.S. Cl. 435/455; 536/23.2

(57) ABSTRACT

The invention concerns a nucleic acid forming part of a cloning vector or designed to be inserted in a cloning vector. Said nucleic acid comprises at least a functional nucleotide sequence, which is needed for cloning and/or expression and is coupled with at least one sequence for identifying type II restriction endo nucleases (REII), so that the cleavage site of the REII concerned is inside said functional nucleotide sequence. The invention also relates to a method using said nucleic acid.

The invention relates to a nucleic acid in a cloning vector or one that is suitable for incorporation into a cloning vector, the nucleic acid having at least one functional sequence necessary for cloning and/or expression.

Cloning vectors are characterized by containing a unique cleavage site for a restriction endonuclease in a region of the vector which is not essential to either the propagation of the vector or for the survival of the host cell. In the prior art, the original cloning vectors with properties such as, for example plasmids pBR322, pAT153 etc. are replaced almost entirely by vectors which contain sequence regions with multitude of successive cleavage sites for various restriction endonucleases, so-called multiple cloning points or polylinkers. Frequently, these cloning sites/polylinker are built into polyfunctional sequence units, wherein the sequence regions also contain other functional sequences, e.g. promoter sequences for RNA-polymerases, with which a transcription of the cloned target-DNA-Fragments (i.e. the DNA fragment which was cloned into the vector) can be realized; termination sequences and polyadenylization signals, or sequences, which facilitate the purification of the protein expressed from the cloned target DNA fragment.

The restriction endonucleases of type II (hereinafter abbreviated as REII) which are commonly used for cloning possess the property that the specific DNA recognition sequence (generally 4-8 base pairs) are at the same time also their specific cleavage site, and that this recognition sequence is point-symmetric with respect to their center and frequently is also a palindrome. That is, when viewing the two strands, the same reading sequence results from both reading directions, which results, that upon cleaving the respective DNA sequence, independent of the source of the DNA, the same compatible DNA ends are generated.

A typical example of an REII is represented by EcoRI. Its recognition sequence is the six base-sequence ^{5′}GAATTC^{3′} on both strands (meaning palindromic), and its cleavage site is located on each strand of the double stranded DNA in 5′ 3′ direction, each time after G and before A:

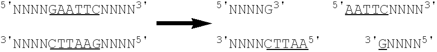

When joining together a cut open cloning vector (upper case letters) and the target DNA fragment to be cloned (lower case letters) which were both produced with EcoRI, the recognition sequence and the cleavage site of this enzyme are again regenerated, specifically at both ends of the respective target-DNA fragments.

| | |
|---|---|
| 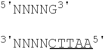 | 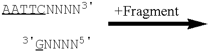 |

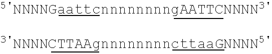

Of course this does not apply only for the EcoRI, but for all REII with palindromic recognition and cleavage sites.

One of the drawbacks of the regular REII is demonstrated when cloning several target DNA fragments with identical ends in a defined sequence into a vector. Since upon cloning of the first target DNA fragment, the recognition sequence and the cleavage site of the respective REII is regenerated two times, namely at both ends of the target DNA fragment, it is not possible, without more, in a subsequent cut, to specifically reopen only one of the two cleavage sites in order to insert a second target DNA fragment. In such a case, those skilllled innthe arts normally tries to conduct a partial digest. However, this approach results in undesirable cleavage products and thus, later also undesirable cloning products.

The principle of these conventional cloning methods by means of REII is schematically illustrated in FIG. 1, wherein the undesired cloning products are shown as shaded gray.

From the schematic representation according to FIG. 1, it is shown that even when cloning-in a second target DNA fragment, the search for the desired correct cloning product proves to be inefficient with respect of time and input. When three, four or more target DNA fragments are to be cloned-in, the conventional method cannot be carried out as a practical matter, even if those skilled in the art uses supplementary methods such as for example overlapping PCRs, double digest and similar.

A further group of restriction endonucleases also known in the prior art, although much less utilized, are the restriction endonucleases of type IIs (hereinafter abbreviated as REIIs) These restriction endonucleases differ from the REII on the one hand in that their recognition sequence (normally 4-8 base pairs) are usually not symmetrical and are thus also not a palindrome, and on the other hand, they differ in that the cleavage site and the recognition sequence are not necessarily identical, but frequently the cleavage site is situated at a defined distance from the recognition sequence. The following are possible for the positions and type of the cleavage site.

a.) Both DNA stands are cut at a defined base distance outside of the recognition sequence (“outside cutter”):

A typical example known in the prior art for such an “outside cutter” is the REIIs AarI. Its recognition sequence is the 7-base sequence ^{5′}CACCTGC^{3″} on one of the DNA strands, respectively ^{5′}GCAGGTG^{3′} on the complementary DNA strand and its cleavage site is located between the fourth and the fifth optional base, which follows in the 5′ 3′ direction the recognition sequence ^{5′}CACCTGC^{3′}, respectively between the eighth and ninth optional base, which is located in the direction 5′ 3′ before the recognition sequence ^{5′}GCAGGTG^{3′}.

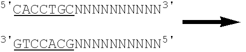

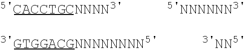

b.) Cleaving is carried out on one strand in a defined base distance outside the recognition sequence; on the other strand, at a defined position within the recognition sequence. (“partial outside cutter”).

An example for this from the prior art is the REIIs BseNI:

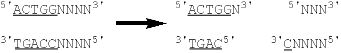

c.) Both strands are being cleaved at defined points within the recognition sequence.

An example for this from the prior art is the REIIs BssSI:

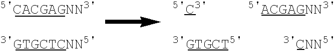

Although not belonging to the REIIs, a further group of restriction endonucleases exhibits similar properties as REIIs, in particular, the “homing nucleases”. These differ from the REIIs foremost by their (normally substantially) longer recognition sequence of normally 8-20 base pairs.

A typical example for a “homing nuclease” commonly known in the prior art is the enzyme I-HmuII (“outside nicker”) with a recognition sequence of 24 bases.

AGTAATGAGCCTAACGCTCAACAANNNNNNNNNNNNNNNNNNNNNNNNNNNNNNNNNNNNNNNN
TCATTACACGGATTGCGAGAAGTTNNNNNNNNNNNNNNNNNNNNNNNNNNNNNNNNNNNNNN_{N}N

Since “outside nicker” normally produces at its cleavage site only a single strand break, by utilizing a combination of at least two of such enzymes, new advantageous cleavage sites for cleaving both nucleic acid strands can be realized.

When cleaving a DNA with an “outside cutter” or “outside nicker” the recognition sequence of the respective enzyme at one of the so obtained DNA fragments remains intact (while being destroyed in the analog case when utilizing an REII for cleaving), and the two ends of the fragments obtained by cutting are normally not compatible with other fragments obtained with the same enzyme and therefore can be re-linked directly only among each other (directly reciprocal). For this reason, these “outside cutter”/“outside nicker” are unsuitable for “conventional” cloning, and to date other areas of use are hardly known.

One of the few examples for the application of such an “outside cutter” is described in the 2000/01 catalog of the company “Fermentas AB, Vilnius, Lithuania)” pages 196/197. “Outside cutter” for cutting of primer sequences in PCR is applied there in order to obtain the ends of a directional cloning in conventional vectors. The methods known in the prior art for directed synthesis of nucleic acid molecules by means of REIIs suffer from several drawbacks. Many of the methods require the application of further complex enzymatic reactions, such for example, fill-in reactions by means of polymerases, cleavage or repair of certain nucleic acid regions by means of nucleases or, the modification of nucleic acids by means of modified enzymes known to those skilled in the art, such for example phosphatases. Moreover, with the known methods it is frequently not possible to link several nucleic acids together in directional manner.

It is an object of the present invention, to provide a useful functional sequence region for cloning and/or expression, without the afore-stated drawbacks when utilizing REIIs for splicing several target DNA fragments, as well as the non-specific cutting of REIIs. It is a further object to provide a method for nucleic acid synthesis without the necessity of complex enzymatic reactions.

As a solution to these objects, a nucleic acid is provided in a cloning vector or one suitable for incorporation into a cloning vector, wherein the nucleic acid has at least one functional nucleotide sequence necessary for cloning and/or expression and which is characterized in that the functional nucleotide sequence is coupled with at least one REIIs-recognition sequence respectively “homing nuclease” recognition sequence in such a manner that the cleavage site of the respective REIIs, respectively the “homing nuclease”, is located within the functional nucleotide sequence. With this nucleic acid it is possible for those skilled in the art to selectively access at least one of the existing recognition sequences of the same REII and/or at least one of the other existing functional sequences in single and repeated succession. The nucleic acid is in particular a DNA or an RNA.

The term “functional nucleotide sequence necessary for cloning and expression” here includes specifically any type of REII cleavage site, but in addition also the RNA-polymerase promoter sequences, functional sequences of the REIIs or the “homing nucleases” itself and other sequences or, partial sequences with certain known functions that are known to those skilled in the art such as for example:

promoter sequences, enhancer, silencer, termination sequences, polyadenylization sequences (regulatory sequences)

genes, open reading frames

protein and RNP-binding sites

sequences which facilitate the purification of the protein expressed from the cloned fragment.

The term “coupled” in the context as used here comprises the following meanings:

that the recognition sequence and the cleavage site of the REIIs respectively the “homing nuclease” are within the functional sequence area necessary for the cloning and/or expression, that is, partial sequence of a nucleotide sequences suitable for cloning target DNA fragments into it or otherwise functional, and

that the cleavage site is situated within a functional sequence area necessary for the cloning and/or expression, that is, partial sequence of a nucleotide sequence suitable for cloning into it DNA fragments or otherwise functional, but the recognition sequence is located outside of the functional sequence area necessary for cloning and/or expression (within the vector sequence).

If necessary, it is unimportant that the recognition sequence or the cleavage site or other functional nucleotide sequences are present only in fragmented form and is being joint together for a specific application by methods known to those skilled in the art in order to generate the nucleic acid according to the present invention.

In a preferred embodiment of the nucleic acid according to the present invention, the properties of REII and REIIs in particular are coupled to functional units, which can each be selectively accessed. Each of the recognition sequences (inter alia in the respective nucleic acid), which can be present also in multiples of the REII can be assigned one ore more of any REIIs whose recognition sequence—as is common for REIIs—is preserved during or after the restriction process, and whose cleavage site is located within the respective REII recognition and cleaving sequence—and if so desired—can be identical with the REII cleavage site. As a result, this REII cleavage site can be specifically and selectively accessed, that means, opened by means of the respective enzyme (REIIs), and in case the cleavage sites of REII and REIIs are identical, then the opening (cutting open) produces the normal cohesive DNA-ends typical for the REII.

This preferred embodiment is used in order to join together serially and linearly several desired nucleic acid fragments in any type of succession, without the need for further enzymatic steps. The orientation of the inserted nucleic acid fragments can be determined by methods known to those skilled in the art, such as for example asymmetrical restriction analysis or sequencing.

The present invention includes also a method for the serial or linear joining of nucleic acids fragments by using the nucleic acid according to the present invention, wherein the method comprises the following steps:

(i) providing a nucleic acid according to the present invention, where the cleavage site of REIIs, respectively the “homing nuclease” is located within the a REII recognition—and cutting sequence and may be identical with the REII cleavage site,

(ii) opening the nucleic acid according to the present invention with the respective REIIs or REII,

(iii) inserting of a first nucleic acid fragment, which is provided at the 5′—as well as also at the 3′ end, with an end compatible with the REII of the nucleic acid according to the invention.

(iv) opening the nucleic acid according to he invention with the respective REIIs (not REII), and

(v) inserting of a second nucleic acid fragment which is provided at the 5′—as well as at the 3′ end with an end that is compatible with the REII of the nucleic acid according to the invention,

wherein the steps (iv) to (v) for inserting of further nucleic acid fragments can be repeated as desired.

The method according to the present invention is further illustrated in example 1.

According to the present invention, one or several such REIIs-coupled REII cleavage sites can be incorporated into one and the same nucleic acid, wherein in the majority of applications each single REIIs/REII combination is present only one time in the nucleic acid. For special application (directional cloning etc.) multiple couplings can be of advantage, that is, the coupling of a REIIs with various REII cleavage sites, respectively, the coupling of various REIIs with one REII cleavage site.

Object of the invention is thus in particular a family of functional DNA cloning and expression sequences each with different combinations from REIIs recognition sequences and REII cleavage sequences.

The REIIs/homing nuclease recognition sequences may be coupled either in lieu of, as a supplemental or in combination with the coupling of the recognition sequences of REII in the same manner as with other functional partial sequences of a polyfunctional sequence unit (of a polyfunctional polylinker), wherein the “functional partial sequence” can be any chosen functional sequence (promoter, enhancer, silencer, protein binding site etc.).

The invention can thus also be applied for the coupling of REIIs with promoter sequences of RNA polymerases. These promoter sequences of RNA polymerases occur, if at all, normally also in multiples in the polyfunctional polylinker, and in many applications should be only one for the activation of a specific purpose. In accordance with the present invention, it is possible, to inactivate single targeted promoter sequences by cleaving with each of the REIIs or “homing nuclease” coupled thereto, so that only one desired promoter sequences remains functional for application of the respective RNA-polymerase. Among others, one advantage is that of several sequentially coupled target DNA fragments (ideally from an entire gene), both sense- and anti-sense transcripts can be produced from one and the same construct with the same RNA-polymerase.

In this context, the present invention includes a method for the selective inactivation of promoter sequences comprising the following method steps:

(i) providing of a nucleic acid according to the present invention, where

a) the cleavage site of the REIIs respectively, the “homing nuclease” is located within the promoter sequence,

b) the promoter sequence is present two times in opposing direction, wherein the two promoter sequences can be identical or different, and

c) both promoter sequences are coupled with at least each of an REIIs (REIIs-A, REIIs-B) and wherein REIIs-A and REIIs-B are different;

(ii) opening the nucleic acid with optionally REIIs-A and/or REIIs-B.

The method according to the invention is further illustrated in example 3. When examining a successful inactivation of a promoter sequence, numerous methods are available to those skilled in the art, such as for example in-vitro-transcription.

The functional nucleotide sequence can also be a recognition sequence of a REIIs or a “homing nuclease”. By means of such a nucleic acid according to the invention it is possible for those skilled in the art to construct entire switching systems.

The nucleic acid according to the invention, which can be a DNA or an RNA can be produced not only directly (as an original construct) but also indirectly, for example as DNA by means of reverse transcriptase from an RNA (as transcription construct).

In a further preferred embodiment, the nucleic acid of the present invention is coupled to carrier material. Among others, that has the advantage, that the so obtained cloning and/or expression products are directly available for further automated treatment or other analyses. The present invention includes all carrier materials suitable for coupling nucleic acids. Examples of these are agarose, silica compounds, polystyrene compounds, teflon-acrylamid, polypropylene, nylon, sephacryl, latex, paramagnetic particles, nanoparticles, and cellulose derivatives. Coupling of the nucleic acid is carried out by methods known to those skilled in the art, and can be carried out in either covalent or non-covalent manner. If desired, the end product can also be separated from the carrier material. Independent of the carrier material—certain enzymes, chemical and high and low-molecular substances, as well as impact of light of predetermined wavelength and temperature dependence are suitable for this purpose.

Object of the present invention, aside from the already described nucleic acids, are also cloning vectors containing such nucleic acids, as well as cells containing one or more such cloning vectors.

It is desirable for the present invention, that the cloning vector utilized for the incorporation into the nucleic acid according to the present invention, contains no further recognition or cleavage sites for REIIs or “homing nucleases” which are contained in the nucleic acid.

The present invention also covers a kit which includes at least a nucleic acid according to the invention, optionally coupled to a carrier material, at least one suitable REIIs, optional in combination with one or more REII, a ligase and if applicable other suitable enzymes und suitable buffers.

### DESCRIPTION OF THE DRAWINGS

FIG. 1:

Cloning of nucleic acid fragments by means of type II restriction nucleases

In part a, the cloning of the fragment A is described, in part b, the additional insertion of fragment B is described. Closed scissors represent any type of type-II restriction endonuclease, black boxes with block arrow represent the recognition- and cutting sequence of the REII. Boxes A and B represent the target DNA to be cloned.

FIG. 2:

Cloning of nucleic acid fragments by means of combinations of type II- and type IIs restriction endonucleases

In part a, the cloning of fragment B is described, in part b, the additional insertion of a fragment A is described. Closed scissors represent any type II restriction endonuclease, open scissors represent any type of Type-IIs restriction endonuclease or “homing nuclease”. Combined shaded/black boxes with black arrow represent the recognition sequence of the REIIs respectively the “homing nuclease” (shaded boxes), coupled with the cutting sequence of any type of Type-II-restriction endonuclease (black box). Boxes A and B is the target DNA to be cloned.

FIG. 3

REIIs/homing nuclease, coupled with functional sequences

The numerals 1 and 2 designate the recognition sequences of Type-IIs restriction endonucleases, wherein 1 and 2 can be identical or different, A represents a functional sequence (i.e. a promoter sequences, see example 3), B is an inserted sequence (for example, a gene).

FIG. 4:

Functional sequence coupled with REIIs in combination with REIIs/REII couplings

The numerals 1 and 2 designate the recognition sequences of type IIs-restriction endonucleases, wherein 1 and 2 can be identical or different, A represents a functional sequence (i.e. the promoter sequence, see example 3), B can be a polylinker from REIIs/“homing nuclease” recognition sequences, coupled with REII-cutting sequences.

FIG. 5:

Several functional sequences coupled with REIIs, in combination with several REIIs/REII couplings

The numerals 1 to 5 designate recognition sequences of Type IIs-restrictions endonucleases, wherein 1 to 5 can be partially or completely identical or partially or completely different, A to E are any functional sequences, a to c are different polylinker.

FIG. 6:

Examplary structure of a polyfunctional DNA cloning site (polylinker) with several REIIs/“homing nuclease” controllable REII cutting sites

The recognition sequence of each REIIs is framed, the recognition and cutting sequence of the so coupled REII is shaded gray, the arrows starting from the frames designate the cleavage site of the REIIs.

The present invention is illustrated further through the following examples:

### EXAMPLE 1

Construction of a nucleic acid in one respectively for a cloning vector with an REIIs AarI coupled EcoRI-(REII-) cleavage site.

Starting components are any type of plasmid, which contains at least one EcoRI-cleavage site, and the REIIs AarI. The REIIs AarI is known to be characterized by the recognition sequence on one strand which is ^{5′}CACCTGC^{3′} and on the complementary strand correspondingly ^{5′}GCAGGTG^{3′} and that its cleavage site on the first strand is located between the recognition sequences fourth and fifth bases following ^{5′}CACCTGC^{3′} in the direction 5′ 3′ and on the complementary strand between the eighth and the ninth base before the recognition sequences ^{5′}GCAGGTG^{3′} in direction 5′ 3′.

AarI:

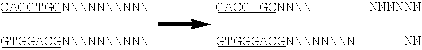

On the plasmid the EcoRI-cleavage site/recognition sequence is localized. Starting from the EcoRI recognition sequence, the recognition sequence for the REIIs AarI is inserted before the EcoRI sequence at a distance of three bases. This incorporation is carried out with methods that are known to those skilled in the art. After the incorporation the respective EcoRI- and the AarI cleavage site are superposed, that is, they are locally identical. Due to the superposition, the respective EcoRI cleavage site can be specifically opened with the REII EcoRI and also with the REIIs AarI resulting in cohesive ends typical for an EcoRI-cut.

| AarI and EcoRI in the nucleic | | |
|---|---|---|
| acid according to the invention | | Cut with the AarI or EcoRI |
| AarI: | AarI EcoRI | AarI |

| | |
|---|---|
| 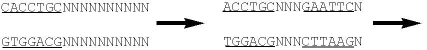 | 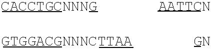 |

The target DNA fragment is cloned into this opened cleavage site, which exhibits the specific EcoRI cutting ends and which was for example obtained through cleaving with EcoRI from another source. This target EcoRI fragment is represented in the following schemata by means of lower case letters:

| AarI | AarI EcoRI EcoRI |
|---|---|
| 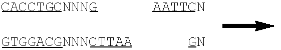 | 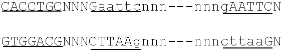 |

In order to selectively insert a second EcoRI-target DNA fragment before this EcoRI-target DNA fragment, a cut has to be carried out with AarI, since only when applying this REIIs, the specific opening of only one cleavage site, namely the EcoRI cleavage site associated with AarI recognition sequence (in the schematic presentation—to the left) is realized, while with the EcoRI both available cleavage sites would have been opened.

| | Cleavage with AarI |
|---|---|
| AarI EcoRI EcoRI | AarI EcoRI |
| 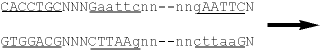 | 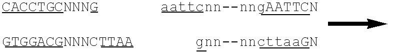 |

AarI EcoRI EcoRI EcoRI

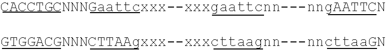

In the same manner, a third, fourth and a further EcoRI-fragment can be selectively inserted and in principle any of a number DNA fragments with EcoRI ends in any desired sequence can be cloned into a nucleic acid, or respectively into the cloning vector provided therein for subsequent expression.

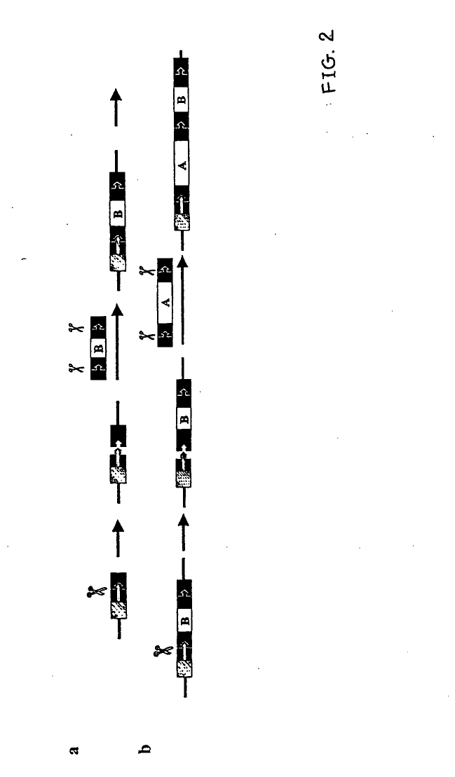

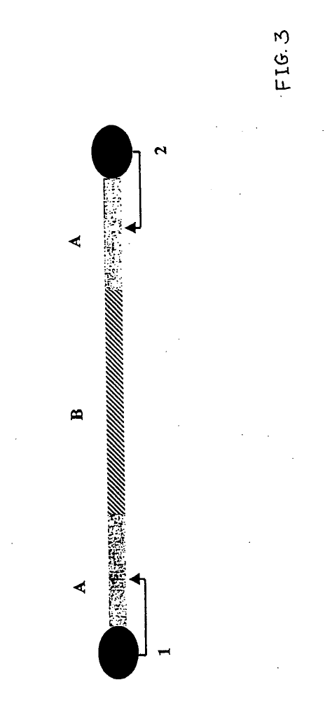

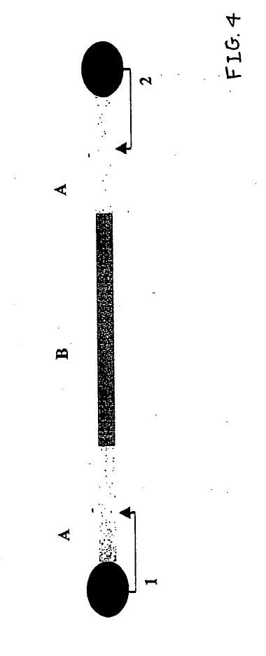

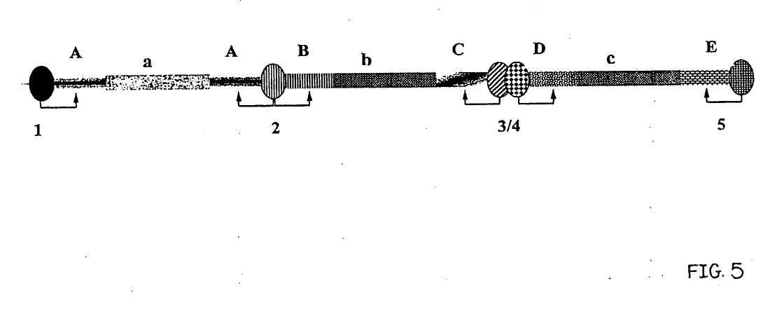

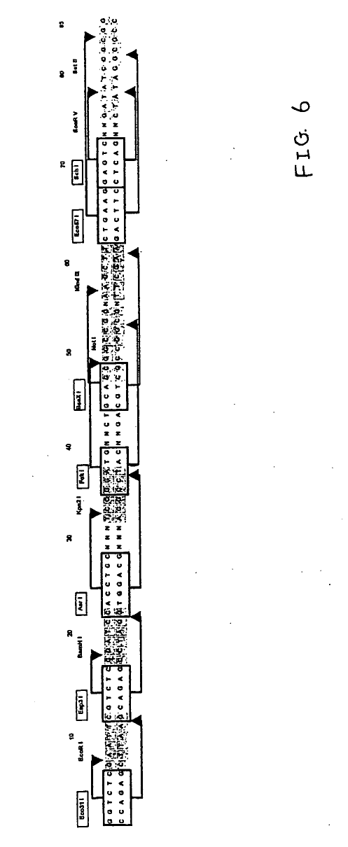

In lieu of the restriction endonucleases EcoRI and AarI which are given here as an example, in principle any type of combination of a REIIs and REII can by utilized. A schematic representation of the principles of the construction according to the present invention is illustrated in FIG. 2.

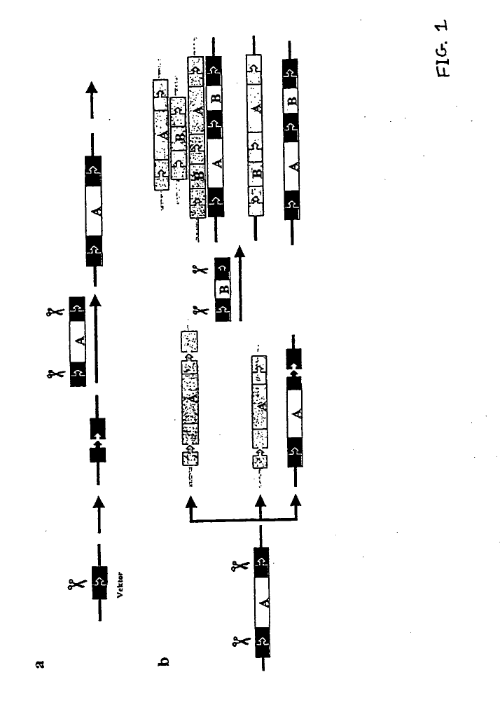

The comparison of the illustrations according to FIG. 1 and FIG. 2 shows the direct advantage of the present invention, namely the unwanted cleaving- and cloning products thereby saving time and effort in searching for the desired product, in particular when linking more than two target DNA fragments.

### EXAMPLE 2

Structure of polyfunctional DNA cloning site (polylinker) with several REIIs/“homing nucleases” controlled REII cleavage sites.

The structure of one of the nucleic acids according to the present invention in the embodiment as a polyfunctional DNA cloning site (polylinker) with several REII cleavage sites controlled by REIIs/“homing nuclease” is schematically illustrated in FIG. 6. The recognition sequence of each REIIs is framed, the recognition- and cleavage sequence of the REII coupled therewith is in shaded gray, the arrows starting from the framing designate the cleavage site of the REII.

### EXAMPLE 3

Promoter sequences for a DNA polymerase for specific control with REIIs AarI and Eco57I.

Starting components are a plasmid, in which the promoter sequence for an RNA-polymerase, e.g. RNA polymerase T7 is configured two times in opposite direction, as well as recognition sequences for the REIIs AarI and Eco57I. The properties of AarI are amply described in example 1. The REIIs Eco57I is characterized in that its recognition sequences on one strand is ^{5′}CTGMG^{3′} and on the complementary strand correspondingly ^{5′}CTTCAG^{3′}, and wherein its cleavage site is on the first strand in direction 5′ 3′ between the sixteenth and seventeenth base following the recognition sequence ^{5′}CTGAAG^{3′} and on the complementary strand is located between the fourteenth and the fifteenth base in direction 5′ 3′ before the recognition sequence ^{5′}CTTCAG^{3′}.

AarI:

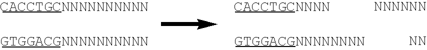

Eco57I:

| | |
|---|---|
| 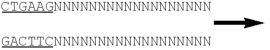 | 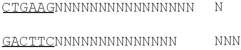 |

Plasmid:

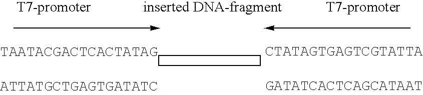

Starting from both of the promoter sequences the recognition sequences for the REIIs AarI and Eco57I are inserted at a suitable distance by means of methods known to those skilled in the art. After the insertion, the promoter sequences and the restriction cleavage sites are overlapping in such a way, that according to the following illustration, the “left” promoter is controllable exclusively by AarI and the “right” promoter exclusively by Eco57I in that each of the enzymes opens the DNA in the respective one promoter sequence, thereby destroying the promoter activity and thus permitting but only one transcription of each of the other promoters.

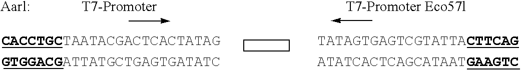

Cleaving with AarI:

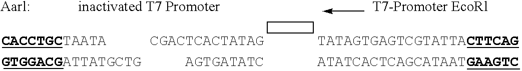

Cleaving with Eco57I:

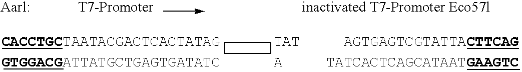

The coupling according to the invention of a REIIs/“homing nuclease” with the functional DNA sequence can be represented in a general way as shown in FIG. 3.

It is understood that both constructs, the coupling of REIIs/“homing nuclease” in form of a single unit or in form of a polyfunctional DNA cloning site (of a polylinker) as well as coupling of REIIs/“homing nuclease” with other functional sequences can be linked in accordance with the present invention also with a single vector, as is for example schematically represented in FIG. 4.

In corresponding manner, the present invention provides also the construction of DNA cloning and expression sequences, wherein a functional sequence is coupled multiple times or different functional sequences with different REIIs/“homing nucleases” and wherein these REIIs/“homing nucleases”-coupled functional sequences are associated with various REIIs/“homing nucleases”-coupled REII cleavage sites. Thus, depending upon the desired constructs, a multitude of combinations is possible as shown in accordance with the schematic illustration in FIG. 5.

1. Nucleic acid in a cloning vector or suitable for incorporation into a cloning vector with at least one functional nucleotide sequence necessary for cloning and/or expression thereof, characterized in that the nucleic acid or at least its functional nucleotide sequence is coupled with at least one REIIs-recognition sequence or “homing nuclease” recognition sequence, so that the cleavage site of the respective REIIs or the “homing nuclease” is located within the functional nucleotide sequence.

2. Nucleic acid according to claim 1, characterized in that the nucleic acid or at least its functional nucleotide sequence is the recognition sequence and/or cleavage site of a restriction endonuclease Type II (REII).

3. Nucleic acid according to claim 1, characterized in that it has several REIIs-coupled REII cleavage sites.

4. Nucleic acid according to claim 1, characterized in that it has a REIIs coupled with different REII cleavage sites, or several different REIIs coupled with one REII cleavage site.

5. Nucleic acid according to claim 1, characterized in that the nucleic acid or at least a functional nucleotide sequence thereof is the recognition sequence of a REIIs or a “homing nuclease”

6. Nucleic acid according to claim 1, characterized in that the nucleic acid or at least the functional nucleotide is the promoter sequence of an RNA polymerase.

7. Nucleic acid according to claim 1, characterized in that the nucleic acid or at least a functional nucleotide sequence thereof is the recognition sequence of a DNA binding protein or RNP particle.

8. Nucleic acid according to claim 1, characterized in that the nucleic acid or at least a functional nucleotide sequence thereof is an enhancer sequence.

9. Nucleic acid according to claim 1, characterized in that the nucleic acid or at least a functional nucleotide sequence thereof is a silencer sequence.

10. Nucleic acid according to claim 1, characterized in that the nucleic acid or at least a functional nucleotide sequence thereof is a termination sequence.

11. Nucleic acid according to claim 1, characterized in that the nucleic acid or at least a functional nucleotide sequence thereof is a polyadenylization sequence.

12. Nucleic acid according to claim 1, characterized in that the nucleic acid or at least a functional nucleotide sequence thereof is a gene or an open reading frame.

13. Nucleic acid according to claim 1, characterized in that the nucleic acid or at least a functional nucleotide sequence thereof is another regulatory sequence.

14. Nucleic acid according to claim 1, characterized in that the nucleic acid or at least a functional nucleotide sequence thereof is a sequence which facilitates the purification the protein expressed from the cloned fragment.

15. Nucleic acid according to claim 1, characterized in that it is a component of a cloning vector.

16. Nucleic acid according to claim 1, characterized in that it is coupled to a carrier material.

17. Nucleic acid according to claim 16, wherein the carrier material is selected from the group consisting of agarose, silica compounds, polystyrene compounds, teflon-acrylamid, polypropylene, nylon, sepharcryl, latex, paramagnetic particles, nanoparticles and cellulose derivatives.

18. Method for serial and linear linkage of nucleic acids fragments by means of the nucleic acid according to the invention, the method comprising the following steps:
(i) providing a nucleic acid according to claim 1, wherein the cutting site of REIIs or the “homing nuclease” is located within the REII recognition- and cutting sequence and optionally is identical with the REII cutting site,
(ii) opening the nucleic acid according to the invention with the respective REIIs or REII,
(iii) inserting of a first nucleic acid fragment which has, at the 5′ end as well as also at the 3′ end, an end compatible with the REII of the nucleic acid according to the invention,
(iv) opening of the nucleic acid according to the invention with the respective REIIs (not REII), and
(v) inserting a second nucleic acid fragment, which has, at the 5′ end as well as also at the 3′ end, an end compatible with the REII of the nucleic acid according to the invention,
wherein the steps (iv) to (v) for inserting further nucleic acid fragments an optionally be repeated as desired.

19. Method for the selective inactivation of promoter sequences, the method comprising the following steps:
(i) providing a nucleic acid according to claim 6, wherein
a) the cleavage site of REIIs or the “homing nuclease” is located within the promoter sequence,
b) the promoter sequence is provided two times in opposite direction, wherein the two promoter sequences can be identical or different, and
c) both promoter sequences coupled with a least one of each REIIs (REIIs-A, REIIs-B) where REIIs-A is different from REIIs-B, and
(ii) opening the nucleic acid with optionally REIIs-A and/or REIIs-B.

20. Cloning vector, which contains a nucleic acid according to claim 1.

21. Cloning vector according to claim 20, characterized in that it contains no further recognition—or cleavage sites for the REIIs or “homing nuclease” occurring in the said nucleic acid.

22. Cells containing a cloning vector, or a nucleic acid according to claim 1.

23. Kit, comprising at least a nucleic acid according to claim 1, at least a suitable REIIs, optionally in combination with one or more REII, a ligase, as well as optionally additional suitable enzymes and suitable reaction buffers.
